(19) United States
(12) Patent Application Publication
(10) Pub. No.: US 2025/0197569 A1
(43) Pub. Date: Jun. 19, 2025

(54) IMPRESSION MATERIALS WITH IMPROVED RHEOLOGICAL PROPERTIES

(71) Applicant: KULZER GMBH, Hanau (DE)

(72) Inventors: Tobias GEISSLER, Gruendau (DE); Andreas GRUNDLER, Muenzenberg (DE); Natalie KAPALA, Frankfurt am Main (DE); Roman HAMBURG, Hungen (DE); Peter DEUTZMANN, Rommerskirchen (DE)

(21) Appl. No.: 18/984,520

(22) Filed: Dec. 17, 2024

(30) Foreign Application Priority Data
Dec. 19, 2023 (DE) 102023135715.8

(51) Int. Cl. C08G 77/12; A61C 9/00; C08G 77/20; C08K 3/36; C08K 7/00

(52) U.S. Cl. CPC C08G 77/12; A61C 9/0006; C08G 77/20; C08K 3/36; C08K 7/00; C08K 2201/003

(57) ABSTRACT

The invention relates to a multi-component system for producing a dental impression material, comprising in two or more separate components: i) one or more crosslinkable (co)polymers, ii) one or more components of a crosslinker system for crosslinking the crosslinkable (co)polymers, and iii) one or more fillers, wherein the multi-component system comprises at least one platelet-shaped filler, wherein the platelet-shaped filler has a first form factor F1=Dₒᵣₜₕₒ/Dₘₐₓ in the range of 0.3 to 1.0 and a second form factor F2=Dₘₐₓ/H of 3 or more, wherein Dₘₐₓ is the maximum platelet diameter in the platelet plane, wherein Dₒᵣₜₕₒ is the largest platelet diameter orthogonal to Dₘₐₓ in the platelet plane, and wherein H is the average platelet thickness orthogonal to the platelet plane.

The invention relates to a multi-component system for producing a dental impression material, a dental impression material produced therefrom and the use of a platelet-shaped filler as a filler in dental impression materials. The use of a corresponding multi-component system for producing a dental impression material is also disclosed.

Techniques for taking dental and jaw impressions continue to play an important role in modern dentistry and in the everyday work of dental technicians. For example, the relevant structures in the patient's jaw are depicted using so-called impression materials. These impression materials are usually provided as multi-component systems of two or more components, which are mixed by the user immediately before use in order to obtain the actual impression material. The multi-component systems are designed in such a way that the mixing of the individual components leads to a curing of the impression material, resulting in a comparatively solid and hard negative form of the structures depicted, which can be used in subsequent processing steps, for example in the manufacture of dental components.

Information on the general technical background is disclosed, for example, in EP 0613926 B1, EP 1946740 A2, US 2005/0239958 A1, EP 2219585 B1 or U.S. Pat. No. 6,599,974 B1.

Nowadays, high demands are placed on corresponding multi-component systems and the dental impression materials that can be produced from them. Many of the desired properties of such multi-component systems relate to the processability of the individual components or the dental impression material. For many of the relevant aspects, there are conflicts of objectives of competing properties that cannot be optimized independently of each other and for which there is a need to find the most advantageous solution to the conflict of objectives. An example of such a conflict of objectives is the competition between a sufficiently long processing time of the mixed impression material, which gives the user enough time to take the desired impression, and advantageous curing kinetics, which minimizes the treatment time for the patient as much as possible.

An important conflict of objectives also exists between the rheological properties, in particular the viscosity and flow behavior, of the components or the not yet fully cured impression material on the one hand and the mechanical properties of the cured dental impression material on the other hand. The individual components of the multi-component system should have an advantageously low viscosity so that they can be easily prepared from the corresponding containers and mixed together efficiently.

The dental impression material obtained from the components is also subject to high demands on viscosity and flow behavior during the processing time. It is desirable that the impression materials are sufficiently stable during application so that they do not leak in the patient's mouth, but flow under slight pressure and can accurately form all the desired details, e.g., the prepared teeth and the sulcus. Favorable flow behavior is particularly important when inserting the tray into the patient's mouth or when injecting around teeth or tooth stumps so that detailed impressions can be taken. These properties play a particularly important role in one-sided impression techniques such as the monophase impression technique. Good flow behavior can also be advantageous for impression materials used as bite registration materials, since these ideally remain stable on the dental arch after application, but at the same time must allow the upper and lower jaws to bite together under low counterpressure without causing a bite shift.

The desired rheological properties are in many respects contradicted by the demands placed on the mechanical properties of the cured dental impression materials. For these, a relatively high final hardness is usually desired, wherein the resulting elastomeric materials should also have an advantageous elongation at break, which in many cases represents a particular challenge, in particular with silicone-based impression materials, since in these a high final hardness is often accompanied by a reduced elongation at break. The realization of these desired properties usually requires the use of high filler contents. However, such high filler contents usually have a negative influence on the rheological properties of the components or the impression material that has not yet fully cured.

The primary object of the present invention was to eliminate or at least mitigate the disadvantages of the prior art.

In particular, it was an object of the present invention to provide a multi-component system for producing a dental impression material, which system advantageously resolves the conflict of objectives between the rheological properties of the components or the not yet fully cured impression material on the one hand and the mechanical properties on the other hand.

In this respect, it was an object of the present invention that the multi-component system to be specified should make it possible to design the individual components with an advantageously low viscosity, so that they can be squeezed out of a cartridge in particular with a low squeezing force and can be easily mixed with one another.

In addition, it was an object of the present invention that the multi-component system to be specified should be convertible into a dental impression material by mixing the components, which is sufficiently stable, but at the same time has advantageous flow behavior with which precise and detailed impressions can be obtained, in particular in difficult-to-access regions.

Furthermore, it was an object of the present invention that the multi-component system to be specified should be convertible into a dental impression material by mixing the components, which should have advantageous mechanical properties after curing, in particular a high final hardness and an advantageous elongation at break. In this respect, it was desirable that the solution to be specified should be applicable for a wide range of filler contents, in particular for high total filler contents.

Against this background, it was an object of the present invention that the solution to be specified should have as little adverse influence as possible on the curing kinetics of the dental impression materials. In addition, it was desirable that the solution to be specified should not impose any additional demands on the devices and methods necessary for the production of the multi-component systems to be specified.

It was a desirable requirement that the multi-component systems to be specified should be manufacturable in the most time- and cost-efficient manner possible and that the solution found should not require the use of materials that are potentially harmful to health and/or the environment.

It was a further object of the present invention to provide a dental impression material produced from the multi-component system to be specified, which has excellent processing properties and at the same time advantageous mechanical properties. In this respect, it was a secondary object of the present invention to specify a use of the multi-component system to be specified for the production of a dental impression material.

In addition, it was an object of the present invention to specify a use for a platelet-shaped filler in dental impression materials.

The inventors of the present invention have now found that the objects described above can surprisingly be achieved if at least one platelet-shaped filler is used as a filler in a multi-component system for producing a dental impression material, as defined in the claims.

Surprisingly, the use of such platelet-shaped fillers makes it possible to obtain multi-component systems for the production of dental impression material, which advantageously resolves the conflict of objectives between the rheological properties of the components or the not yet fully cured impression material on the one hand and the mechanical properties on the other hand, wherein good processing properties can be achieved for the use of the dental impression material as well as for the further processing of the impressions produced with it. Advantageously, high filler contents are also possible. In addition, the use of appropriate platelet-shaped fillers, particularly in silicone-based impression materials, allows favorable tear properties to be achieved despite a high final hardness. In addition, the use of such platelet-shaped fillers advantageously has no or no noticeable negative influence on the curing kinetics of the dental impression materials.

The above-mentioned objects are thus solved by the subject matter of the invention as defined in the claims. Preferred designs according to the invention are apparent from the dependent claims and the following embodiments.

Such embodiments, which are hereinafter designated as preferred, are combined in particularly preferred embodiments with features of other embodiments designated as preferred. Combinations of two or more of the embodiments referred to below as particularly preferred are thus very particularly preferred. Also preferred are embodiments in which a feature of one embodiment designated as preferred to any extent is combined with one or more other features of other embodiments designated as preferred to any extent. Features of preferred dental impression materials and uses are derived from the features of preferred multi-component systems.

Insofar as both specific amounts or proportions of an element, for example for the platelet-shaped fillers or the crosslinkable (co)polymers, and preferred embodiments of the element are disclosed below, the specific amounts or proportions of the preferably configured elements are also disclosed. In addition, it is disclosed that, given the corresponding specific total amounts or total proportions of the elements, at least some of the elements can be preferably configured and, in particular, that preferably configured elements within the specific total amounts or total proportions can in turn be present in the specific amounts or proportions.

The invention relates to a multi-component system for producing a dental impression material, comprising in two or more separate components:
i) one or more crosslinkable (co)polymers,
ii) one or more components of a crosslinker system for crosslinking the crosslinkable (co)polymers, and
iii) one or more fillers,
wherein the multi-component system comprises at least one platelet-shaped filler,
wherein the platelet-shaped filler has a first form factor F1=Dₒᵣₜₕₒ/Dₘₐₓ in the range of 0.3 to 1.0 and a second form factor F2=Dₘₐₓ/H of 3 or more,
wherein Dₘₐₓ is the maximum platelet diameter in the platelet plane, wherein Dₒᵣₜₕₒ is the largest platelet diameter orthogonal to Dₘₐₓ in the platelet plane, and wherein H is the average platelet thickness orthogonal to the platelet plane.

Dental impression materials and the multi-component systems used for their production as such are in principle comprehensively known to a person skilled in the art from the prior art.

In accordance with the understanding of a person skilled in the art and the usual procedure in the field of technology, the multi-component systems according to the invention are defined by the components contained in the multi-component system, which are presented in two or more separate components of the multi-component system, for example in different containers. Individual components of the multi-component system can also be present in two or more, or all, of the components. The production of the dental impression material from the components of the multi-component system is usually carried out by mixing the components, which can be promoted or caused, for example, by a suitable mixing device, wherein suitable mixing devices, which can be designed, for example, as mixing syringes, are commercially available from numerous manufacturers. A multi-component system according to the invention is preferred because of easy handling of only two components, wherein the multi-component system is a two-component system which comprises the components i), ii) and iii) divided into two separate components. Particularly relevant in practice is a multi-component system according to the invention, wherein the separate components of the multi-component system are present in different containers, preferably in separate tubes or in separate chambers of a cartridge for use in a mixing device, preferably in separate chambers of a cartridge for use in a mixing syringe.

These components of the multi-component system defined above are each used as “one or more” in accordance with the understanding of a person skilled in the art. The term “one or more” refers, in accordance with industry practice, to the chemical nature of the compounds in question and not to their quantity. For example, the multi-component system may comprise only one type of crosslinkable (co)polymers, which would mean that the multi-component system comprises a plurality of the corresponding molecules.

Insofar as mass fractions are specified within the scope of the invention, the combined mass fractions of one or more components are specified in the usual manner in the industry, thereby expressing that the mass fraction of the correspondingly designed components taken together meets the corresponding criteria, wherein in the absence of other information, the total mass of the multi-component system forms the reference system.

The multi-component system according to the invention comprises one or more crosslinkable (co)polymers in at least one component, preferably in two or more of the components, particularly preferably in all components. The term “(co)polymers” expresses the fact that these are higher molecular weight compounds that can be produced from a large number of monomer units. In accordance with the understanding of a person skilled in the art, the boundaries between polymers and oligomers are not sharply defined. In the scope of the present invention, the term “(co)polymers” also comprises (co)oligomers for reasons of concise designation, wherein the use of (co)polymers in the narrower sense is preferred.

The (co)polymers to be used according to the invention are crosslinkable. In accordance with the understanding of a person skilled in the art, this means that they have one or more chemical functionalities through which crosslinking of the (co)polymers is possible. The prior art provides a very large number of different basic systems for dental impression materials, which rely on different crosslinkable (co)polymers. The type of crosslinking mechanism of the chemical compounds required for this depends on the chemical nature of the (co)polymers, wherein a variety of different crosslinking concepts are used in the prior art. Functionally, however, these very different combinations of crosslinkable (co)polymers and crosslinking concepts always have in common that the crosslinkable (co)polymers used are crosslinked by a suitable crosslinking system, utilizing the respective chemical functionalities. Even if, for example, radical mechanisms are also conceivable, a multi-component system according to the invention is preferred, wherein the multi-component system is a multi-component system for producing a dental impression material by means of addition crosslinking or condensation crosslinking, preferably by addition crosslinking.

The present invention relates, in accordance with the understanding of a person skilled in the art, in particular to an innovation with regard to filler systems for dental impression materials. In this respect, the inventors have found that the advantageous effects of the present invention can be realized substantially with all basic systems formed of (co)polymer and corresponding crosslinking chemistry, which leads to the assumption that the advantageous effects are based in particular on physical or mechanical interactions and that the chemical interaction with the fillers plays a lesser role. This broad applicability of the teaching according to the invention can in itself be seen as an advantage. However, it is also particularly advantageous in this respect that, as a result of this finding, there is no longer any need to individually define the large number of possible combinations of (co)polymers and the crosslinking systems coordinated with them. Rather, when implementing the invention, a person skilled in the art can resort to the basic systems for dental impression materials known from the prior art and optimize them by using the specific platelet-shaped fillers. In light of this finding, the multi-component system according to the invention comprises one or more components which serve to crosslink the (co)polymers during subsequent mixing and which are combined as a crosslinker system within the scope of the present invention. The crosslinker system basically comprises the components which are known from the prior art for the crosslinking of the corresponding (co)polymers.

The crosslinking can, for example, take place as homo-crosslinking between the (co)polymers, for example by exploiting an inherent reactivity through the use of a suitable activator and/or catalyst or by initiating a radical polymerization with an initiator, for example. Additionally or alternatively, it is also possible for hetero-polymerization to occur, in which the (co)polymers are at least partially linked via further compounds, so-called crosslinkers, wherein the reactivity between (co)polymers and crosslinker compounds can, if required, be promoted or brought about by activators, catalysts and/or initiators. Depending on the underlying polymer system, a multi-component system according to the invention is preferred, wherein the multi-component system comprises as components of the crosslinker system: ii.a) one or more crosslinker compounds for covalently crosslinking the crosslinkable (co)polymers, and/or ii.b) one or more catalyst compounds, and/or ii.c) one or more activator compounds, and/or iv.d) one or more initiator compounds, in particular photoinitiators.

Because of the wide variety of possible basic systems that are compatible with the present invention, it is very difficult to specify preferred mass fractions for the components. However, in the opinion of the inventors, a multi-component system according to the invention is relevant for many applications with regard to the crosslinker compounds, wherein the combined mass fraction of the one or more crosslinking compounds is in the range of 0.25 to 20%, preferably in the range of 1 to 15%, particularly preferably in the range of 2 to 10%, in relation to the total mass of the multi-component system. When using catalysts and similar compounds, however, a multi-component system according to the invention is typically used additionally or alternatively, wherein the combined mass fraction of the one or more catalyst compounds and/or the activator compounds and/or the initiator compounds is in the range of 0.01 to 0.5%, preferably in the range of 0.02 to 0.1%, in relation to the total mass of the multi-component system.

A person skilled in the art understands that the crosslinker system, with regard to its distribution among the components of the multi-component system, is subject to the proviso that the crosslinker system should not lead to crosslinking and thus to curing of the components before mixing the components—or at least not to any significant extent. Accordingly, for substantially all embodiments, it is preferred that none of the components of the multi-component system comprises both the crosslinkable (co)polymers and all components of the crosslinker system.

In addition, it is possible that the crosslinker system comprises different components which can realize crosslinking of the (co)polymers via different crosslinking mechanisms. This makes it possible to realize multi-stage crosslinking kinetics and to adapt the curing behavior specifically to the respective application requirements.

A particularly important group of (co)polymer basic systems for impression materials are the so-called polyether impression materials, in which polyethers, which can be functionalized with aziridino groups, for example, are used and which can be polymerized using an acid or another cationic initiator, for example. Corresponding polyether impression materials are known, for example, from EP 0613926 B1, EP 1946740 A2 or EP 2219585 B1. In principle, corresponding polyether impression materials are considered advantageous with regard to many relevant properties, in particular because these polyether impression materials usually have a high final hardness with good to very good elongation at break. In combination with the use of the specific platelet-shaped fillers provided for in the invention, this results in particularly high-performance impression materials. For many applications, a multi-component system according to the invention is preferred, wherein the one or more (co)polymers are selected from the group consisting of functionalized polyethers, preferably polyethers with aziridino groups, wherein the crosslinker system preferably comprises at least one acid.

Another class of basic systems that are highly relevant in the field of technology are the so-called silicone impression materials, as disclosed, for example, in US 2005/0239985 A1. However, these silicone impression materials, which are known in principle and suitable for many applications, are sometimes perceived as disadvantageous, particularly in comparison to polyether impression materials, since the desired combination of a high final hardness with good to very good elongation at break is often not, or at least not easily, achieved in these materials. The inventors have found that, surprisingly, when the invention is adapted to silicone impression materials, not only the viscometric properties and the flow behavior can be improved, but that the choice of filler also shows an improvement in the conflict of objectives between hardness and elongation at break observed for silicone impression materials. Because of this synergistic improvement of a large number of application-relevant parameters, the inventors believe it is particularly advantageous to implement the present invention using a silicone impression material as the basic system. Accordingly, a multi-component system according to the invention is preferred, wherein the one or more (co)polymers are selected from the group consisting of functionalized organopolysiloxanes.

For example, a multi-component system according to the invention is preferred for the silicone impression materials, wherein the one or more (co)polymers are selected from the group consisting of organopolysiloxanes comprising allyl groups and organopolysiloxanes comprising vinyl groups, preferably with two or more allyl groups or vinyl groups, particularly preferably terminal allyl groups or vinyl groups, in the molecule. Additionally or alternatively, a multi-component system according to the invention is preferred, wherein the crosslinker system comprises one or more crosslinker compounds selected from the group consisting of organohydrogenpolysiloxanes, wherein the crosslinker compound and the one or more (co)polymers are preferably present at least partially in the same component of the multi-component system. Additionally or alternatively, a multi-component system according to the invention is also preferred, wherein the crosslinker system comprises one or more catalyst compounds selected from the group consisting of noble metal catalysts, preferably platinum catalysts.

As an alternative to the above-mentioned silicone impression materials, systems in which crosslinking occurs via condensation between OH groups and with alkoxysilane groups are particularly suitable. A multi-component system according to the invention is preferred, wherein the one or more (co)polymers are selected from the group consisting of organopolysiloxanes comprising hydroxyl groups. Additionally or alternatively, a multi-component system according to the invention is preferred, wherein the crosslinker system comprises one or more crosslinker compounds selected from the group consisting of silicic acid esters, preferably organopolysiloxanes comprising alkoxysilane groups, wherein the crosslinker compound and the one or more (co)polymers are preferably not present in the same component of the multi-component system. Additionally or alternatively, a multi-component system according to the invention is also preferred, wherein the crosslinker system comprises one or more catalyst compounds selected from the group consisting of organometallic compounds, preferably organotin compounds, for example dioctyltin oxide, dibutyltin oxide and complexes of these organotin compounds with monocarboxylic acids, in particular dilaurates.

According to the invention, the multi-component system comprises one or more fillers, wherein at least one of these fillers must be a platelet-shaped filler, wherein two or more different types of platelet-shaped filler can also be used. Qualitatively, these platelet-shaped fillers can also be called “flakes.” In other words, this is a multi-component system according to the invention, wherein the platelet-shaped filler has the form of flakes.

In the scope of the present invention, the platelet-shaped fillers are characterized by two form factors, wherein a filler which satisfies these form factors is regarded as a platelet-shaped filler.

The first form factor F1=Dₒᵣₜₕₒ/Dₘₐₓ, in other words, defines the ratio of the platelet width to the platelet length and, as a result of the defined range, expresses that the platelet-shaped filler is not a rod-shaped or needle-shaped filler. A multi-component system according to the invention is preferred, wherein the platelet-shaped filler has a first form factor F1=Dₒᵣₜₕₒ/Dₘₐₓ in the range of 0.35 to 1.0, preferably in the range of 0.4 to 1.0, particularly preferably in the range of 0.45 to 1.0, most preferably in the range of 0.5 to 1.0.

The second form factor F2=Dₘₐₓ/H describes the ratio between the platelet length and the platelet thickness and expresses that the platelets are comparatively flat. A multi-component system according to the invention is preferred, wherein the platelet-shaped filler has a second form factor F2=Dₘₐₓ/H of 4 or more, preferably 5 or more, particularly preferably 6 or more. Additionally or alternatively, a multi-component system according to the invention is preferred, wherein the platelet-shaped filler has a second form factor F2=Dₘₐₓ/H in the range of 3 to 90, preferably in the range of 4.5 to 60, particularly preferably in the range of 5 to 30.

In their own experiments, the inventors have succeeded in identifying particularly suitable dimensions for the platelet-shaped fillers with regard to the absolute size ranges, with which the conflict of objectives between the rheological properties, in particular the viscosity and flow behavior, of the components or the not yet fully cured impression material on the one hand and the mechanical properties of the cured dental impression material on the other hand can be resolved particularly advantageously.

A multi-component system according to the invention is preferred, wherein the platelet-shaped filler has a maximum platelet diameter in the platelet plane Dₘₐₓ in the range of 1 to 600 μm, preferably in the range of 2 to 500 μm, particularly preferably in the range of 3 to 400 μm, very particularly preferably in the range of 4 to 200 μm, especially preferably in the range of 5 to 100 μm. Additionally or alternatively, a multi-component system according to the invention is preferred, wherein the platelet-shaped filler has an average platelet thickness orthogonal to the platelet plane H in the range of 0.2 to 20 μm, preferably in the range of 0.5 to 15 μm, particularly preferably in the range of 0.7 to 10 μm, very particularly preferably in the range of 1.0 to 6.0 μm, and/or wherein the platelet-shaped filler has an average platelet thickness orthogonal to the platelet plane H in the range of 2 to 10 μm, preferably in the range of 2.5 to 8 μm, particularly preferably in the range of 3 to 6 μm.

With regard to the size distribution of the platelet length, a multi-component system according to the invention is preferred, wherein the platelet-shaped filler has a volume-related D50 value of the maximum platelet diameter in the platelet plane D50ₘₐₓ in the range of 5 to 50 μm, preferably in the range of 6 to 40 μm, particularly preferably in the range of 7 to 30 μm, and/or wherein the platelet-shaped filler has a volume-related D50 value of the maximum platelet diameter in the platelet plane D50ₘₐₓ in the range of 10 to 40 μm, preferably in the range of 12 to 35 μm, particularly preferably in the range of 14 to 30 μm, most preferably in the range of 16 to 25 μm.

Without wishing to be bound by this theory, the inventors assume, on the basis of the experiments carried out, that it is the specific morphology of the platelet-shaped fillers that causes the advantageous effects of the present invention on the rheological properties. In this respect, it can be seen as an advantage of the present invention that it is in principle very flexible with regard to the chemical nature of the platelet-shaped fillers to be used, so that, for example, platelet-shaped plastics can also be used, which can be advantageous, for example, with regard to the total weight of the multi-component systems. However, according to the inventors, some of the mechanical properties that are influenced by filling with filler, in particular the hardness, are more dependent on the material properties of the filler, wherein, according to the inventors, it is in principle advantageous to use inorganic materials instead of mostly relatively soft plastics. An example is a multi-component system according to the invention, wherein the platelet-shaped filler is an organic or inorganic, preferably an inorganic, filler. An additional or alternative example is a multi-component system according to the invention, wherein the platelet-shaped filler is an amorphous filler.

The inventors consider the use of glass flakes to be particularly preferred, as they result in multi-component systems and dental impression materials that can be produced therefrom with a particularly advantageous property profile. Accordingly, a multi-component system according to the invention is preferred, wherein the platelet-shaped filler consists of a glass to a mass fraction of 90% or more, preferably 95% or more, particularly preferably 99% or more, very particularly preferably substantially completely, wherein the glass is preferably selected from the group consisting of silicate glasses and borate glasses, preferably borosilicate glasses, particularly preferably aluminoborosilicate glasses.

The inventors consider it particularly preferred if a silicon-containing platelet-shaped filler, for example a silicate glass, is used as the platelet-shaped filler. Particularly in combination with silicone impression materials, particularly advantageous improvements in the ratio of final hardness to elongation at break are achieved.

The inventors have succeeded in identifying particularly suitable proportions for the content of platelet-shaped fillers, wherein these can be related both to the total mass of the multi-component system and to the total mass of the filler system used. A multi-component system according to the invention is preferred, wherein the multi-component system comprises the platelet-shaped filler in a combined mass fraction in the range of 0.2 to 75%, preferably in the range of 0.5 to 50%, particularly preferably in the range of 1 to 40%, very particularly preferably in the range of 2 to 30%, particularly preferably in the range of 3 to 25%, extremely preferably in the range of 5 to 20%, in relation to the total mass of the multi-component system. Additionally or alternatively, a multi-component system according to the invention is also preferred, wherein the multi-component system comprises the platelet-shaped filler in a combined mass fraction of 5% or more, preferably 15% or more, particularly preferably 25% or more, very particularly preferably 45% or more, particularly preferably 75% or more, potentially preferably 95% or more, in relation to the total mass of fillers in the multi-component system.

In addition to the platelet-shaped filler(s), other fillers can also be provided in the multi-component systems according to the invention, in particular using those conventional fillers which are also used in the prior art for dental impression materials. A multi-component system according to the invention is preferred, wherein the multi-component system comprises, in addition to the at least one platelet-shaped filler, at least one further filler which is not a platelet-shaped filler, wherein the further filler is preferably selected from the group consisting of rock flours, silicates, carbonates, sulfates, quartz flours, cristobalite flours, precipitated amorphous silicon dioxide or pyrogenic amorphous silicon dioxide, starch, particularly preferably consisting of quartz flours, cristobalite flours, precipitated amorphous silicon dioxide or pyrogenic amorphous silicon dioxide.

A multi-component system according to the invention is preferred, wherein the multi-component system has a total filler content of 40% or more, preferably 45% or more, particularly preferably 50% or more, very particularly preferably 55% or more, in relation to the total mass of the multi-component system. Additionally or alternatively, a multi-component system according to the invention is preferred, wherein the combined mass fraction of all fillers in the multi-component system is in the range of 20 to 80%, preferably in the range of 30 to 75%, particularly preferably in the range of 40 to 70%, in relation to the total mass of the multi-component system.

The inventors have succeeded in identifying particularly advantageous mass ratios between the platelet-shaped fillers and the other fillers. A multi-component system according to the invention is preferred, wherein the quotient of the combined mass fraction of the platelet-shaped fillers divided by the combined mass fraction of the further fillers is in the range of 0.01 to 1.0, preferably in the range of 0.015 to 0.7, particularly preferably in the range of 0.02 to 0.6, very particularly preferably in the range of 0.025 to 0.5.

The inventors have found that the use of the platelet-shaped filler has a beneficial effect on the rheological properties of the components of the multi-component system, regardless of which components of the crosslinking system are also present in this component. Accordingly, the inventors consider it particularly advantageous to use the platelet-shaped filler in several, preferably all, components of the multi-component system. Accordingly, a multi-component system according to the invention is preferred, wherein the multi-component system comprises the at least one platelet-shaped filler in two or more, preferably in three or more, particularly preferably in all separate components. Particularly preferred is a multi-component system according to the invention, wherein the platelet-shaped filler is present in two or more of the separate components of the multi-component system, preferably in all separate components of the multi-component system, wherein the mass fraction of the platelet-shaped filler in relation to the mass of the component in the separate components preferably differs by 80% or less, preferably 60% or less, wherein the mass fraction of the platelet-shaped filler in relation to the total mass of filler in the respective component preferably differs by 20% or less, preferably by 10% or less, particularly preferably by 5% or less.

According to the inventors' findings, a particularly advantageous adjustment of the rheological properties during processing and the mechanical properties of the cured impression material can be achieved in particular by combining two or more different platelet-shaped fillers, which can preferably be used in the same component, particularly preferably in all components. Accordingly, a multi-component system according to the invention is preferred, wherein the multi-component system comprises two or more platelet-shaped fillers, preferably exactly two, particularly preferably in a ratio of the mass fractions in the range of 4:1 to 1:4, very particularly preferably in the range of 2:1 to 1:2, particularly preferably in the range of 1.5:1 to 1:1.5, wherein the two or more platelet-shaped fillers preferably differ with respect to at least one aspect which is selected from the group consisting of the form factor F1, the form factor F2, the maximum platelet diameter Dₘₐₓ, the average platelet thickness H and the chemical composition, particularly preferably with regard to the chemical composition and at least one further aspect which is selected from the group consisting of the form factor F1, the form factor F2, the maximum platelet diameter Dₘₐₓ and the average platelet thickness H.

With regard to the distribution of the constituents among the components of the multi-component system, preference is given, for example, to a multi-component system according to the invention, wherein the multi-component system comprises a first component A, wherein component A contains:
A.i.) one or more crosslinkable (co)polymers,
A.ii.a) one or more crosslinker compounds, and
A.iv) one or more fillers, wherein component A preferably comprises at least one platelet-shaped filler as a filler.

In this respect, additionally or alternatively, preferably additionally, a multi-component system according to the invention is preferred, wherein the multi-component system comprises a second component B, wherein component B contains:
B.i.) one or more crosslinkable (co)polymers,
B.ii.b) one or more catalyst compounds and/or activator compounds and/or initiator compounds, preferably catalyst compounds,
and optional but preferably:
B.iii) one or more fillers, wherein component B preferably comprises at least one platelet-shaped filler as a filler.

With regard to the distribution of the constituents among the components of the multi-component system, preference is also given alternatively, for example, to a multi-component system according to the invention, wherein the multi-component system comprises a first component A, wherein component A contains:
A.i.) one or more crosslinkable (co)polymers, and
A.iv) one or more fillers, wherein component A preferably comprises at least one platelet-shaped filler as a filler.

In this respect, additionally or alternatively, preferably additionally, a multi-component system according to the invention is preferred, wherein the multi-component system comprises a second component B, wherein component B contains:
B.ii.c) one or more activator compound compounds,
and optional but preferably:
B.iii) one or more fillers, wherein component B preferably comprises at least one platelet-shaped filler as a filler.

It can be seen as an advantage of the multi-component systems according to the invention that they are very flexible with regard to the presence of other typical additives and can be specifically tailored to specific application requirements by using appropriate additives. For many applications, a multi-component system according to the invention is preferred, wherein the multi-component system additionally comprises:
iv) one or more further additives, wherein the further additives are selected from the group consisting of thixotropic agents, pigments, dyes, hydrophilic agents, stabilizers, inhibitors, odorants and flavorings.

The invention also relates to a dental impression material produced or producible by mixing the separate components of the multi-component system according to the invention. In this context, the use of a multi-component system according to the invention for producing a dental impression material by mixing the separate components of the multi-component system is also disclosed.

The invention also relates to the use of a platelet-shaped filler as a filler in multi-component systems for producing a dental impression material to improve the rheological properties of the components of the multi-component system or the impression material,
wherein the platelet-shaped filler has a first form factor F1=Dₒᵣₜₕₒ/Dₘₐₓ in the range of 0.3 to 1.0 and a second form factor F2=Dₘₐₓ/H of 3 or more,
wherein Dₘₐₓ is the maximum platelet diameter in the platelet plane, wherein Dₒᵣₜₕₒ is the largest platelet diameter orthogonal to Dₘₐₓ in the platelet plane, and wherein H is the average platelet thickness orthogonal to the platelet plane.

The invention and preferred embodiments of the invention are explained and described in more detail below with reference to accompanying FIGURE. In the FIGURE:

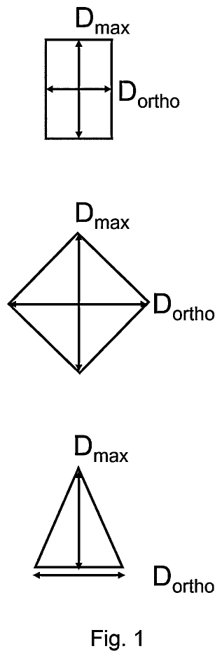

FIG. 1 is a schematic visualization of the reference values used to calculate the form factors of the platelet-shaped fillers.

FIG. 1 shows in a schematic visualization of three exemplary, abstracted forms of platelet-shaped fillers in the top view of the platelet plane how the maximum platelet diameter Dₘₐₓ and the largest orthogonal plate diameter Dₒᵣₜₕₒ are determined.

The invention and preferred embodiments of the invention are additionally explained and described in more detail below with reference to experiments.

### A. Investigations:

The following tests were carried out as part of the experiments.

The rheological properties of the individual components (base paste BP and catalyst paste KP) of the samples were determined. The measurements of the rheological properties were carried out using a rheometer from Thermo-Fischer. The plate/cone measuring geometry was used. For these measurements, the cone rotates on a substance that is located on the stationary plate. The measuring gap between cone and plate as well as the temperature (23° C.) remained constant throughout the entire measurement. Viscosity describes the resistance of a liquid to a shear force and is therefore known as toughness. The yield point describes a force that is required to make a substance flow.

To measure the viscosity and the yield point, the shear rate of the cone is increased from 0.00 1/s to 5.00 1/s over a period of 3.00 min. The viscosity in Pa*s is measured at a shear rate of 3.00 1/s. The yield point in Pa is obtained by interpolation/linear regression of the data points at decreasing shear rate and is marked as axis intercept “a” (resulting from y=a+bx).

The mixing disk was carried out on the mixtures of the base paste BP and the catalyst paste KP according to DIN ISO 4823 2021-06 using an apparatus according to A.1 (two square glass plates approx. 60×60 mm, minimum distance 3 mm, sample quantity 0.5+/−0.02 ml, loading time 5 min, measuring temperature: 23° C.+/−2° C.).

The Shore A hardness of the dental impression materials was determined after 10 min, 1 h and 24 h.

In addition, the elongation at break according to DIN 53504:2017-03 and the E-modulus (bending) according to DIN13903: 2005-10 were determined for the dental impression materials.

In addition, the established “Shark Fin Test” (M. Zenginel et al., Deutsche Zahnärztliche Zeitschrift, 2011; 66 (12), “Shark Fin Test and rheological properties of elastomeric impression materials: A correlation analysis”; DOI 10.3238/dzz.2011.0899) was carried out on the dental impression materials, which visually indicates the flow properties of the impression materials in the mixed state in the form of a fin height.

### A. Sample Preparation—Test Series 1:

In the experiments of the first test series, four different basic formulations GZ1 to GZ4 based on polydimethylsiloxane (vinyl-terminated, CAS: 68083-19-2, total mass fraction in the range of approx. 20 to 30%) and the crosslinker methylhydrogenpolysiloxane (CAS: 68037-59-2; total mass fraction in the range of approx. 5 to 8%) were used, which differ in terms of the additives used and the total mass fractions of the components, in particular in the total filler content, as well as their distribution on the so-called base paste (BP) and a so-called catalyst paste (KP).

The basic formulations GZ1 to GZ4 are suitable model systems which, in the opinion of the inventors, provide an excellent basis for understanding the influence of the fillers, wherein the basic formulations GZ1 to GZ4 provide a good impression of commercially relevant products. Based on these model systems, only the composition of the fillers is changed while maintaining the same total filler content. Against this background, it is advantageous to refrain from providing a more comprehensive list of the components of the basic formulations for reasons of clarity.

The relevant total filler content of the basic formulations GZ1 to GZ4 is listed in Table 1 below.

TABLE 1
Total filler content of the basic formulations GZ1 to GZ4
in relation to the total mass of the basic formulations

| Basic formulation | Total filler content/% |
|---|---|
| GZ1 | 44.01 |
| GZ2 | 44.30 |
| GZ3 | 49.42 |
| GZ4 | 49.46 |

The samples were prepared based on the basic formulations GZ1 to GZ4. The fillers summarized in Table 2 were used.

TABLE 2
Fillers used

| Abbreviation | Note |
|---|---|
| Filler 1 | Platelet-shaped filler formed of aluminoborosilicate |
| | glass, D50ₘₐₓ = 8-12 μm, H = 1.0-1.3 μm, trade name |
| | GF001-10, company: Glassflake Limited |
| Filler 2 | Platelet-shaped filler formed of aluminoborosilicate |
| | glass, D50ₘₐₓ = 18-22 μm, H = 3.5-5.5 μm, trade |
| | name GF005-20, company: Glassflake Limited |
| Filler 3 | Quartz-based filler (cristobalite), D50ₘₐₓ = 2.5 μm; Trade |
| | name Silbond |
| Filler 4 | Filler based on amorphous silicon dioxide (CAS: 7631- |
| | 86-9) |

The samples produced are summarized in Table 3, showing in which total mass fraction the respective fillers are used.

TABLE 3
Filler contents of the samples, all data in mass fractions in %, in
relation to the total mass of the basic formulations - Test series 1

| Sample | Basic formulation | Filler 1 | Filler 2 | Filler 3 | Filler 4 |
|---|---|---|---|---|---|
| V1 | GZ1 | 0 | 0 | 37.56 | 6.45 |
| E1 | GZ1 | 0 | 5 | 32.56 | 6.45 |
| E2 | GZ1 | 0 | 10 | 27.56 | 6.45 |
| V2 | GZ2 | 0 | 0 | 37.85 | 6.45 |
| E3 | GZ2 | 0 | 10 | 27.85 | 6.45 |
| E4 | GZ2 | 10 | 0 | 27.85 | 6.45 |
| E5 | GZ2 | 0 | 10 | 27.85 | 6.45 |
| E6 | GZ2 | 0 | 10 | 27.85 | 6.45 |
| V3 | GZ3 | 0 | 0 | 43.55 | 5.87 |
| E7 | GZ3 | 0 | 18.23 | 25.32 | 5.87 |
| E8 | GZ3 | 18.23 | 0 | 25.32 | 5.87 |
| E9 | GZ3 | 9.11 | 9.11 | 25.32 | 5.87 |
| E10 | GZ3 | 4.55 | 13.66 | 25.34 | 5.87 |
| E11 | GZ3 | 13.66 | 4.55 | 25.34 | 5.87 |
| V4 | GZ4 | 0 | 0 | 43.59 | 5.87 |
| E12 | GZ4 | 18.21 | 0 | 25.38 | 5.87 |
| E13 | GZ4 | 28.21 | 0 | 15.38 | 5.87 |
| E14 | GZ4 | 21.78 | 21.78 | 0 | 5.87 |
| E15 | GZ1 | 0 | 10 + 10 | 27.56 | 6.45 |

With the exception of samples E5 and E6, fillers 1 and 2 were each added to a premixed basic formulation which already contained filler 3. In order to exclude the possibility that the production sequence has a decisive influence on the material properties, in samples E5 and E6, filler 2 was added during the production of the remaining basic formulation and processed completely with the other components.

Sample E15 corresponds to sample E2, with an additional 10% of filler 2 added to obtain a higher total filler content.

In the basic formulations, the amount of filler was distributed between the base paste (BP) and the catalyst paste (KP), wherein approximately 42 to 52% of the total filler is used in the base paste and approximately 51 to 65% of the total filler is used in the catalyst paste. If filler 1 and/or filler 2 were used in the samples, these were divided equally between the base paste and the catalyst paste without changing the relative filler proportions in the pastes (sample E2, for example, contains 5% of filler 2 in KP and BP in relation to the total mass of the formulation). This approach was chosen because it allows a broader distribution of the relative proportions of the filler system to be investigated.

The properties determined on the samples are summarized in Tables 4 to 7.

TABLE 4
Measured values - Test series 1 - Part 1

| Size | V1 | E1 | E2 |
|---|---|---|---|
| Viscosity BP/(Pa*s) | 147.7 | 120.0 | 115.1 |
| Yield point BP/Pa | 243.7 | 181.7 | 167.8 |
| Viscosity KP/(Pa*s) | 121.0 | 94.54 | 79.96 |
| Yield point KP/Pa | 184.8 | 136.5 | 100.2 |
| Mixing disk/mm | 37 | 38 | 38 |
| Shore A (10 min) | 49.8 | 48.2 | 48.3 |
| Shore A (1 h) | 50.0 | 48.8 | 48.7 |
| Shore A (24 h) | 50.5 | 48.9 | 49.3 |
| Elongation at break/% | 188 | 201 | 214 |
| E-modulus | 0.58 | 0.54 | 0.56 |
| (bending)/MPa | | | |
| Shark Fin Test | 6.5 | 8 | 9 |

TABLE 5
Measured values - Test series 1 - Part 2

| Size | V2 | E3 | E4 | E5 | E6 |
|---|---|---|---|---|---|
| Viscosity BP/(Pa*s) | 86.61 | 77.77 | 78.89 | 79.07 | 84.84 |
| Yield point BP/Pa | 132.0 | 107.8 | 110.2 | 112.8 | 123.9 |
| Viscosity KP/(Pa*s) | 126.7 | 81.88 | 84.93 | 89.33 | 94.77 |
| Yield point KP/Pa | 182.3 | 84.71 | 95.04 | 115.6 | 129.3 |
| Mixing disk/mm | 31.5 | 39 | 38 | 38.5 | 38 |
| Shore A (10 min) | 48.0 | 47.7 | 48.8 | 48.6 | 49.6 |
| Shore A (1 h) | 48.0 | 48.0 | 48.7 | 49.1 | 49.8 |
| Shore A (24 h) | 48.4 | 48.2 | 50.3 | 49.4 | 50.0 |
| Elongation at break/% | 188 | 198 | 169 | 172 | 176 |
| E-modulus | 0.47 | 0.44 | 0.56 | 0.48 | 0.51 |
| (bending)/MPa | | | | | |
| Shark Fin Test | 6.9 | 8.2 | 7.5 | 7.5 | 7.17 |

TABLE 6
Measured values - Test series 1 - Part 3

| Size | V3 | E7 | E8 | E9 | E10 | E11 |
|---|---|---|---|---|---|---|
| Viscosity BP/(Pa*s) | 125.9 | 87.08 | 104 | 93.43 | 92.13 | 100.3 |
| Yield point BP/Pa | 213.7 | 125.1 | 155.1 | 134.8 | 134.8 | 145.7 |
| Viscosity KP/(Pa*s) | 257.5 | 106.6 | 131.7 | 112 | 111.5 | 116.1 |
| Yield point KP/Pa | 321.5 | 125.2 | 164.6 | 129.4 | 125.5 | 136.1 |
| Mixing disk/mm | 34.5 | 37 | 36 | 37 | 37 | 36 |
| Shore A (10 min) | 50.1 | 51.2 | 53.1 | 52.2 | 51.7 | 53.7 |
| Shore A (1 h) | 50.2 | 51.6 | 53.3 | 52.2 | 52.0 | 54.1 |
| Shore A (24 h) | 51.6 | 51.7 | 54.4 | 52.9 | 52.8 | 54.4 |
| Elongation at break/% | 213 | 184 | 169 | 178 | 173 | 152 |
| E-modulus (bending)/MPa | 0.35 | 0.66 | 0.71 | 0.69 | 0.59 | 0.68 |
| Shark Fin Test | 3.5 | 6 | 5.4 | 6 | 6.2 | 5.5 |

TABLE 7
Measured values - Test series 1 - Part 4 (n.d. = not determined)

| Size | V4 | E12 | E13 | E14 | E15 |
|---|---|---|---|---|---|
| Viscosity BP/(Pa*s) | 128.6 | 99.56 | 93.29 | 97.69 | 148.6 |
| Yield point BP/Pa | 218.7 | 146.7 | 125.7 | 132.7 | 218.0 |
| Viscosity KP/(Pa*s) | 241.4 | 120.0 | 101.5 | 86.61 | 92.8 |
| Yield point KP/Pa | 268.6 | 122.1 | 81.6 | 46.55 | 106.3 |
| Mixing disk/mm | 35 | 37.5 | 39 | 37 | n.d. |
| Shore A (10 min) | 49.8 | 52 | 53.2 | 50.2 | n.d. |
| Shore A (1 h) | 50.3 | 52.1 | 53 | 50.3 | n.d. |
| Shore A (24 h) | 50.9 | 53.9 | 53.6 | 52.4 | n.d. |
| Elongation at break/% | 211 | 164 | 171 | 238 | n.d. |
| E-modulus | 0.27 | 0.67 | 0.56 | 0.55 | n.d. |
| (bending)/MPa | | | | | |
| Shark Fin Test | 4.06 | 6.21 | 6.29 | 7.07 | 7.5 |

The use of platelet-shaped fillers (fillers 1 and 2) provided for in accordance with the invention surprisingly leads to consistently improved rheological properties with regard to the flowability of the materials and—as a result—to increased mixing disks, in comparison with conventional fillers for impression materials, wherein the advantageous effect is particularly pronounced with increasing proportion of the platelet-shaped filler in the total filler content.

In accordance with this finding, favorable values are achieved for the mixing disk and a significant improvement in the shark fin test. Advantageously, the mechanical properties remain at least at a comparable level or are even improved. In particular, in many cases a favorable property profile is shown in the ratio of the usually competing values between hardness and elongation at break.

The comparison of V1, E1, E2 and E15 shows that higher total filler contents can be achieved with comparable or even slightly improved flow properties through the use of platelet-shaped fillers as provided for in the invention.

### B. Sample Preparation—Test Series 2:

In the experiments of the second test series, further samples were produced starting from a base paste basic formulation BGZ with an initial filler content of a quartz-based filler of 33%. Additional filler was added to the base paste basic formulation. The corresponding compositions are summarized in Table 8.

TABLE 8
Filler contents of the samples, all data in mass
fractions in %, in relation to the total mass of
the base paste basic formulations - Test series 2

| Sample | Filler 1 | Filler 2 | Filler 3 |
|---|---|---|---|
| V5 | 0 | 0 | 34 |
| V6 | 0 | 0 | 35.5 |
| V7 | 0 | 0 | 38 |
| E16 | 1 | 0 | 33 |
| E17 | 2.5 | 0 | 33 |
| E18 | 5 | 0 | 33 |
| E19 | 0 | 1 | 33 |
| E20 | 0 | 2.5 | 33 |
| E21 | 0 | 5 | 33 |

The measured values obtained on the samples are summarized in Table 9.

TABLE 9
Measured values - Test series 2

| Sample | Viscosity BP/(Pa*s) | Yield point BP/Pa |
|---|---|---|
| V5 | 141.1 | 225.3 |
| V6 | 150.4 | 242.9 |
| V7 | 166.0 | 271.7 |
| E16 | 139.1 | 223.5 |
| E17 | 143.4 | 229.0 |
| E18 | 158.4 | 254.0 |
| E19 | 131.9 | 212.6 |
| E20 | 137.6 | 219.4 |
| E21 | 144.9 | 231.9 |

These values also demonstrate that the use of platelet-shaped fillers according to the invention leads to consistently improved flow properties or mixing disks, in comparison with conventional fillers for impression materials, wherein the advantageous effect is particularly pronounced with increasing proportion of platelet-shaped filler in the total filler content, wherein it is again clear that higher total filler contents can be achieved with comparable rheological properties.

1. A multi-component system for producing a dental impression material, comprising in two or more separate components:
i) one or more crosslinkable (co)polymers,
ii) one or more components of a crosslinker system for crosslinking the crosslinkable (co)polymers, and
iii) one or more fillers,
wherein the multi-component system comprises at least one platelet-shaped filler,
wherein the platelet-shaped filler has a first form factor F1=Dₒᵣₜₕₒ/Dₘₐₓ in the range of 0.3 to 1.0 and a second form factor F2=Dₘₐₓ/H of 3 or more,
wherein Dₘₐₓ is the maximum platelet diameter in the platelet plane, wherein Dₒᵣₜₕₒ is the largest platelet diameter orthogonal to Dₘₐₓ in the platelet plane, and wherein H is the average platelet thickness orthogonal to the platelet plane.

2. The multi-component system according to claim 1, wherein the multi-component system comprises the platelet-shaped filler in a combined mass fraction in the range of 0.2 to 75%, in relation to the total mass of the multi-component system.

3. The multi-component system according to claim 1, wherein the multi-component system comprises the platelet-shaped filler in a combined mass fraction of 5% or more, in relation to the total mass of fillers in the multi-component system.

4. The multi-component system according to claim 1, wherein the platelet-shaped filler is an amorphous filler.

5. The multi-component system according to claim 1, wherein the platelet-shaped filler consists of a glass to a mass fraction of 90% or more.

6. The multi-component system according to claim 1, wherein the platelet-shaped filler has a volume-related D50 value of the maximum platelet diameter in the platelet plane D50ₘₐₓ in the range of 5 to 50 μm.

7. The multi-component system according to claim 1, wherein the platelet-shaped filler has a first form factor F1=Dₒᵣₜₕₒ/Dₘₐₓ in the range of 0.35 to 1.0, and/or wherein the platelet-shaped filler has a second form factor F2=Dₘₐₓ/H of 4 or more.

8. The multi-component system according to claim 1, wherein the one or more (co)polymers are selected from a group consisting of functionalized organopolysiloxanes.

9. A dental impression material, produced or producible by mixing the separate components of the multi-component system according to claim 1.

10. A use of a platelet-shaped filler as a filler in dental impression materials to improve the rheological properties of the impression material, wherein the platelet-shaped filler comprises a first form factor F1=Dₒᵣₜₕₒ/Dₘₐₓ in the range of 0.3 to 1.0 and a second form factor F2=Dₘₐₓ/H of 3 or more, wherein Dₘₐₓ is the maximum platelet diameter in the platelet plane, wherein Dₒᵣₜₕₒ is the largest platelet diameter orthogonal to Dₘₐₓ in the platelet plane, and wherein H is the average platelet thickness orthogonal to the platelet plane.
